(19) United States
(12) Patent Application Publication
(10) Pub. No.: US 2025/0037802 A1
(43) Pub. Date: Jan. 30, 2025

(54) System and Methods for Predicting Polymer Properties

(71) Applicant: Georgia Tech Research Corporation, Atlanta, GA (US)

(72) Inventors: Rampi RAMPRASAD, Atlanta, GA (US); Christopher KUENNETH, Atlanta, GA (US)

(21) Appl. No.: 18/595,796

(22) Filed: Mar. 5, 2024

Related U.S. Application Data
(63) Continuation of application No. PCT/US2023/073627, filed on Sep. 7, 2023.
(60) Provisional application No. 63/374,761, filed on Sep. 7, 2022.

(51) Int. Cl. G16C 20/30; G01N 33/44; G16C 20/70

(52) U.S. Cl. CPC G16C 20/30; G01N 33/442; G16C 20/70

(57) ABSTRACT

A method for predicting polymer properties that can include converting chemical fragments from a plurality of first polymers into standardized data strings, separating each of the standardized data strings into one or more tokens, predicting, via a first machine learning algorithm, one or more tokens from each of the standardized data strings, computing, via a processor device, one or more unique fingerprints for each of the standardized data strings, and mapping, via a second machine learning algorithm, one or more properties of the plurality of first polymers and one or more properties of a plurality of second polymers to the one or more unique fingerprints.

### CROSS-REFERENCE TO RELATED APPLICATIONS

This is a Continuation Application of International Application No. PCT/US2023/073627 filed 7 Sep. 2023, which International Application claims the benefit of U.S. Provisional Application Ser. No. 63/374,761, filed 7 Sep. 2022, the entire contents and substance of which are incorporated herein by reference in their entirety as if fully set forth below.

### STATEMENT REGARDING FEDERALLY SPONSORED RESEARCH OR DEVELOPMENT

This invention was made with government support under Grant No. GR10005221 awarded by the Office of Naval Research (ONR), and Grant No. GR00004636 awarded by the National Science Foundation (NSF). The government has certain rights in the invention.

### THE NAMES OF THE PARTIES TO A JOINT RESEARCH AGREEMENT

Not Applicable

### SEQUENCE LISTING

Not Applicable

### STATEMENT REGARDING PRIOR DISCLOSURES BY THE INVENTOR OR A JOINT INVENTOR

Not Applicable

### BACKGROUND OF THE DISCLOSURE

### 1. Field of the Invention

The various embodiments of the present disclosure relate generally to polymer chemical informatics, specifically systems and methods for predicting properties of chemical polymer.

### 2. Description of Related Art

Polymers are an integral part of everyday life and instrumental in the progress of technologies for future innovations. The sheer magnitude and diversity of the polymer chemical space provide opportunities for crafting polymers that accurately match application demands, yet also come with the challenge of efficiently and effectively browsing the gigantic space of polymer systems. The nascent field of polymer informatics allows access to the depth of the polymer universe and demonstrates the potency of machine learning (ML) models to overcome this challenge. ML frameworks have enabled substantial progress in the development of polymer property predictors and solving inverse problems in which polymers that meet specific property requirements are either identified from candidate sets or are freshly designed using genetic or generative algorithms.

Thus, a need exists for systems and methods that can effectively and efficiently traverse the expansive world of polymers to determine properties for varying polymer chemical structures on demand for real-life applications.

### BRIEF SUMMARY

An exemplary of the present invention comprises an integrated end-to-end completely machine-driven polymer informatics pipeline uses an encoder-only transformer chemical language model to replace conventional handcrafted fingerprints. The present invention is more than two orders of magnitude faster than conventional approaches.

Another exemplary of the present invention comprises a method that views polymers as a chemical language of atoms and atom connectivities. With this perspective, language model development techniques adopted by the Natural Language Processing (NLP) community, become applicable. The instant innovations include the creation of a training set of 100 million polymers, the creation of a transformer based architecture specifically to learn the chemical language of polymers, and training this architecture to yield a machine-driven polymer fingerprinting model. These fingerprints can then be used in down-stream machine learning models to build polymer property predictors.

Another exemplary of the present invention comprises a method comprises preprocessing polymer simplified molecular input line end system (“PSMILES”) strings, feeding the preprocessed PSMILES strings to an encoder-only transformer chemical language model, fingerprinting at least a portion of an output of the encoder-only transformer chemical language model, and predicting polymer properties from the fingerprinted output.

In any of the exemplary embodiments, the method is an integrated end-to-end completely machine-driven polymer informatics pipeline.

In any of the exemplary embodiments, the preprocessing comprises canonicalizing the PSMILES strings into standardized data strings, tokenizing at least a portion of the standardized data strings, and masking at least a portion of the tokenized standardized data strings.

In any of the exemplary embodiments, the method further comprises creating a training set of polymers, training the encoder-only transformer chemical language model with the training set, and yielding a machine-driven polymer fingerprinting model for the fingerprinting.

In any of the exemplary embodiments, the preprocessing comprises converting chemical fragments from a set of first polymers into standardized data strings by representing set of first polymers into the PSMILES strings, separating the PSMILES strings into one or more tokens, and predicting, via a first machine learning algorithm, one or more tokens from the PSMILES strings,

In any of the exemplary embodiments, the fingerprinting comprises computing, via a processor device, one or more unique fingerprints for the PSMILES strings, and mapping, via the encoder-only transformer chemical language model, one or more properties of the set of first polymers and one or more properties of set of second polymers to the one or more unique fingerprints.

In any of the exemplary embodiments, the predicting comprises predicting, via encoder-only transformer chemical language model, one or more properties for a new polymer based at least in part on one or more of the properties of the set of second polymers and one or more of the properties of set of second polymers.

In any of the exemplary embodiments, the representing the set of first polymers into the PSMILES strings comprises canonicalizing the PSMILES string to create the PSMILES strings for the set of first polymers.

In any of the exemplary embodiments, the separating the PSMILES strings into one or more tokens comprises parsing through the PSMILES strings using one or more text delimiters comprises tokenizing the PSMILES strings based at least in part on the one or more text delimiters.

In any of the exemplary embodiments, the predicting, via the first machine learning algorithm, the one or more tokens from the PSMILES strings comprises creating a masked portion of one or more of the tokens and an unmasked portion of one or more of the tokens for the PSMILES strings.

In any of the exemplary embodiments, the creating the masked portion and the unmasked portion within the PSMILES strings comprises embedding one or more of the tokens of the unmasked portion with a numerical weight, and predicting the masked portion based on the numerical weight for one or more of the tokens of the unmasked portion.

In any of the exemplary embodiments, the embedding one or more of the tokens of the unmasked portion with a numerical weight comprises passing one or more of the tokens of the unmasked portion through one or more neural encoder layers and one or more neural decoder layers, and updating the numerical weight through one or more of the neural decoder layers and one or more of the neural decoder layers for one or more of the tokens of the unmasked portion.

In any of the exemplary embodiments, the updating the numerical weight through one or more of the neural decoder layers and one or more of the neural decoder layers for one or more of the tokens of the unmasked portion comprises determining a syntactical relationship between one of more of the tokens within the PSMILES strings by creating an attention map for one of more of the tokens, wherein the attention map is configured to plot an attention score for one of more of the tokens.

In any of the exemplary embodiments, the mapping, via encoder-only transformer chemical language model, the one or more properties of the set of first polymers and the one or more properties of set of second polymers to the one or more unique fingerprints comprises receiving an input vector of one of more of the unique fingerprints, and mapping the input vector with the polymer properties via a selector vector, wherein the selector vector is a binary vector configured to represent the polymer properties using a binary number format.

In any of the exemplary embodiments, the method further comprises mapping the polymer properties based at least in part on one of more of the unique fingerprints, and outputting one of more of the polymer properties for one of more of the unique fingerprints by filtering the output of one or more polymer properties based at least in part on one or more search parameters.

Another exemplary embodiment of the present invention comprises a system for predicting polymer properties comprising a processor device configured to convert chemical fragments from a set of first polymers into a set of second polymers different than the first polymers, convert the set of second polymers into the PSMILES strings, separate the PSMILES strings into one or more tokens, and compute one or more unique fingerprints for the PSMILES strings with the encoder-only transformer chemical language model.

In any of the exemplary embodiments, the processor device is further configured to parse through the PSMILES strings using one or more text delimiters, and tokenize the PSMILES strings based at least in part on one of more of the text delimiters.

In any of the exemplary embodiments, the processor device is further configured to train a machine learning algorithm configured to predict one or more tokens of the PSMILES strings.

In any of the exemplary embodiments, the machine learning algorithm is further configured to use natural language processing (NLP) on one of more of the tokens of each of the PSMILES strings, and create a masked portion of one of more of the tokens and an unmasked portion of one of more of the tokens for the PSMILES strings.

In any of the exemplary embodiments, the machine learning algorithm is further configured to embed one of more of the tokens of the unmasked portion with a numerical weight, analyze the numerical weight for one of more of the tokens of the unmasked portion to predict the masked portion.

In any of the exemplary embodiments, the machine learning algorithm is further configured to pass one of more of the tokens of the unmasked portion through one or more neural encoder layers and one or more neural decoder layers, update the numerical weight through one of more of the neural encoder layers and one or more of the neural decoder layers for one of more of the tokens of the unmasked portion, determine a syntactical relationship between one of more of the tokens within the PSMILES strings, and create an attention map for one of more of the tokens, wherein the attention map is a plot of an attention score for one of more of the tokens.

Another exemplary embodiment of the present invention comprises system for predicting polymer properties comprising a processor device configured to receive an input vector, map via a machine learning algorithm each entry of the input vector with a selector vector indicative of polymer properties, and output polymer properties for one or more of the entries of the input vector, wherein one or more of the entries of the input vector is indicative of a unique fingerprint for one or more polymers.

In any of the exemplary embodiments, the machine learning algorithm is a multitask deep neural network.

In any of the exemplary embodiments, the processor device is further configured to filter the output of one or more of the polymer properties based at least in part on one or more search parameters.

In any of the exemplary embodiments, the selector vector is a binary vector configured to represent the polymer properties using a binary number format.

Another exemplary embodiment of the present disclosure provides method for predicting polymer properties that can comprise converting chemical fragments from a plurality of first polymers into standardized data strings, separating each of the standardized data strings into one or more tokens, predicting, via a first machine learning algorithm, one or more tokens from each of the standardized data strings, computing, via a processor device, one or more unique fingerprints for each of the standardized data strings, and mapping, via a second machine learning algorithm, one or more properties of the plurality of first polymers and one or more properties of a plurality of second polymers to the one or more unique fingerprints.

In any of the embodiments disclosed herein, the method may further comprise predicting, via a second machine learning algorithm, the one or more properties for a new polymer based at least in part on the one or more properties of the plurality of first polymers and one or more properties of the plurality of second polymers.

In any of the embodiments disclosed herein, the standardized data strings may comprise a polymer simplified molecular input line end system (“PSMILES”) string.

In any of the embodiments disclosed herein, the method may further include representing the standardized data strings for the chemical fragments from the plurality of first polymers as PSMILES string may comprise canonicalizing each PSMILES string to create the standardized data strings for the plurality of first polymers.

In any of the embodiments disclosed herein, the method may further include separating each of the standardized data strings into one or more tokens may comprise parsing through each of the standardized data strings using one or more text delimiters. The method also includes parsing through each of the standardized data strings using one or more delimiters may comprise tokenizing each of the standardized data strings based at least in part on the one or more text delimiters.

In any of the embodiments disclosed herein, the method may further include predicting, via the first machine learning algorithm, one or more tokens of each of the standardized data strings may comprise creating a masked portion of the one or more tokens and an unmasked portion of the one or more tokens for each of the standardized data strings. The method also includes creating a masked portion and an unmasked portion within each of the standardized data strings may comprise embedding each of the one or more tokens of the unmasked portion with a numerical weight, and predicting the masked portion based on the numerical weight for each of the one or more tokens of the unmasked portion.

In any of the embodiments disclosed herein, the method may further include embedding each of the one or more tokens of the unmasked portion with a numerical weight may comprises passing each of the one or more tokens of the unmasked portion through one or more neural encoder layers and one or more neural decoder layers, and updating the numerical weight through each of the one or more encoder layers and each of the one or more decoder layers for each of the one or more tokens of the unmasked portion.

In any of the embodiments disclosed herein, the method may further include updating the numerical weight through each of the one or more encoder layers and each of the one or more decoder layers for each of the one or more tokens of the unmasked portion may comprise determining a syntactical relationship between the one or more tokens within each standardized data string. The method also includes determining a syntactical relationship between the one or more tokens within each standardized data strings may comprise creating an attention map for the one or more tokens, wherein the attention map can be configured to plot an attention score for each of the one or more tokens.

In any of the embodiments disclosed herein, the method may further include utilizing a second machine learning algorithm to map one or more unique fingerprints to a plurality of polymer properties may comprise receiving an input vector of the one or more unique fingerprints, and mapping the input vector with the plurality of polymer properties via a selector vector. The selector vector may be a binary vector that can be configured to represent the plurality of polymer properties using a binary number format.

In any of the embodiments disclosed herein, the method may further comprise mapping the plurality of polymer properties based at least in part on the one or more unique fingerprints, which may comprise outputting the one or more polymer properties for each of the one or more unique fingerprints. The method also includes outputting the plurality of polymer properties for each of the one or more unique fingerprints may comprise filtering the output of one or more polymer properties based at least in part on one or more search parameters.

Another embodiment of the present disclosure provides a system for predicting polymer properties, the system may comprise a processor that can be configured to convert chemical fragments from a plurality of first polymers into a plurality of second polymers different than the first polymers, convert the plurality of second polymers into standardized data strings, separate the standardized data strings into one or more tokens, and compute a unique fingerprint for each of the standardized data strings.

In any of the embodiments disclosed herein, the standardized data strings may be a plurality of a polymer simplified molecular input line end system (PSMILES) strings.

In any of the embodiments disclosed herein, the processor may be further configured to parse through each of the standardized data strings using one or more text delimiters, and tokenize each of the standardized data strings based at least in part on the one or more text delimiters.

In any of the embodiments disclosed herein, the processor may be further configured to train a machine learning algorithm that can be configured to predict one or more tokens of each of the standardized data strings. The machine learning algorithm may be further configured to use natural language processing (NLP) on the one or more tokens of each of the standardized data strings.

In any of the embodiments disclosed herein, the machine learning algorithm may be further configured to create a masked portion of the one or more tokens and an unmasked portion of the one or more tokens for each of the standardized data strings. The machine learning algorithm is further configured to embed each of the one or more tokens of the unmasked portion with a numerical weight. The machine learning algorithm is further configured to analyze the numerical weight for each of the one or more tokens of the unmasked portion to predict the masked portion.

In any of the embodiments disclosed herein, the machine learning algorithm may be further configured to pass each of the one or more tokens of the unmasked portion through one or more neural encoder layers and one or more neural decoder layers. The machine learning algorithm is further configured to update the numerical weight through each of the one or more encoder layers and each of the one or more decoder layers for each of the one or more tokens of the unmasked portion. The machine learning algorithm may be further configured to determine a syntactical relationship between the one or more tokens within each standardized data string.

In any of the embodiments disclosed herein, the machine learning algorithm may be further configured to create an attention map for the one or more tokens, and wherein the attention map is a plot of an attention score for each of the one or more tokens.

Another embodiment of the present disclosure provides a system for predicting polymer properties, the system may comprise a processor that can be configured to receive an input vector, map via a machine learning algorithm each entry of the input vector with a selector vector indicative of a plurality of polymer properties, and output the plurality of polymer properties for each entry of the input vector. Each entry of the input vector may be indicative of a unique fingerprint for each of a plurality of polymers.

In any of the embodiments disclosed herein, the machine learning algorithm may be a multitask deep neural network. The selector vector may be a binary vector configured to represent the plurality of polymer properties using a binary number format. The processor may be further configured to filter the output of the plurality of polymer properties based at least in part on one or more search parameters.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A is a system flow diagram for a system that receives standardized data inputs representing polymer chemical structures and outputs predicted polymer properties for the standardized data inputs, in accordance with an exemplary embodiment of the present disclosure.

FIG. 1B is an exemplary standardized data string for a polymer chemical structure, in accordance with an exemplary embodiment of the present disclosure.

FIG. 1C is a second system flow diagram for a system that receives standardized data inputs representing polymer chemical structures and outputs predicted polymer properties for the standardized data inputs, in accordance with an exemplary embodiment of the present disclosure.

FIG. 2 is a uniform manifold approximation and projection (UMAP) plots comparing polymer prediction capabilities of handcrafted polymer fingerprints to polymer fingerprints developed by an embodiment of the present technology, in accordance with an exemplary embodiment of the present disclosure.

FIG. 3 is an attention map plot demonstrating how a machine learning algorithm learns to decipher standardized data strings representing polymer, in accordance with an exemplary embodiment of the present disclosure.

FIG. 4 is a table of chemical polymer properties predicted by handcrafted polymer fingerprints versus polymer properties predicted by an embodiment of the present technology, in accordance with an exemplary embodiment of the present disclosure.

FIG. 5 is a method flow chart for predicting polymer properties, in accordance with an exemplary embodiment of the present disclosure.

FIG. 6 is an illustration of an exemplary computing environment, in accordance with an exemplary embodiment of the present disclosure.

FIG. 7 is a plot comparing computation speeds of polymer fingerprints between an embodiment of the present technology and traditional polymer fingerprinting technologies, in accordance with an exemplary embodiment of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

By “comprising” or “containing” or “including” is meant that at least the named compound, element, particle, or method step is present in the composition or article or method, but does not exclude the presence of other compounds, materials, particles, method steps, even if other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described as making up the various elements of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

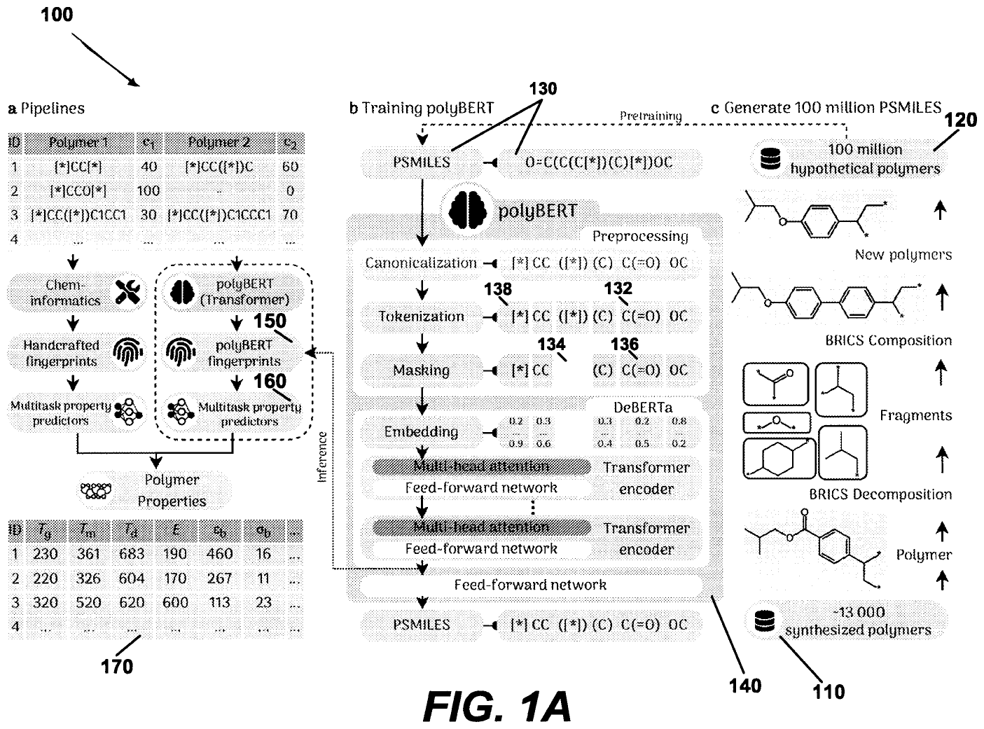

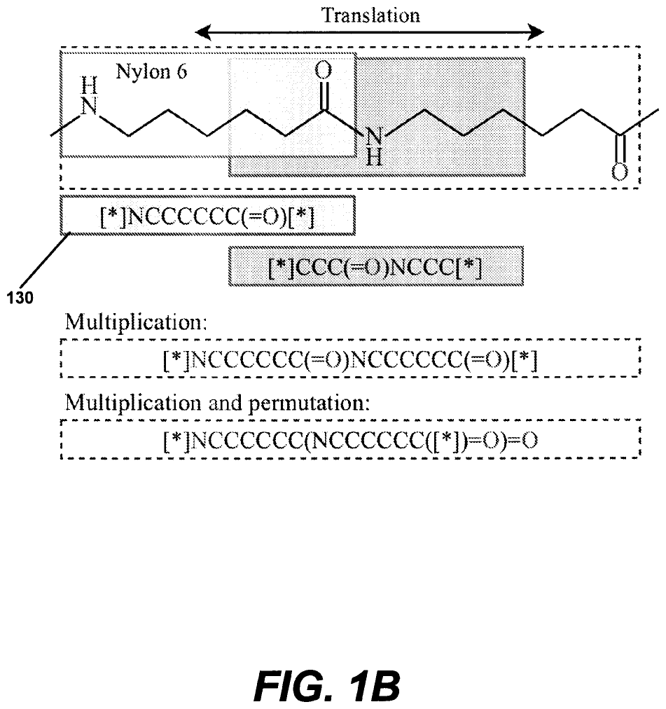

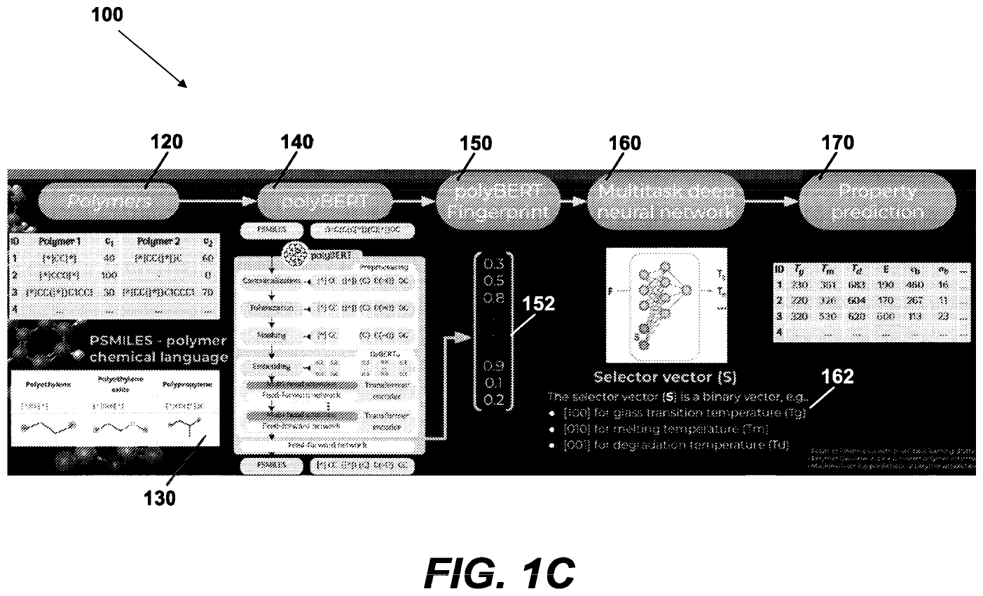

FIGS. 1A and 1C are system flow diagrams for a system 100 to predict polymer properties 170 for chemical polymers. The system 100 can be configured to convert chemical fragments of chemical polymers from a plurality of first polymers 110 into a plurality of second polymers 120. In some embodiments, as shown in FIG. 1A, the plurality of first polymers 110 can include at least approximately 13,000 synthesized polymer structures (e.g., at least approximately 13,500 structures, at least approximately 14,000 structures, at least approximately 14,500 structures, at least approximately 15,000 structures, at least approximately 15,500 structures, at least approximately 16,000 structures, at least approximately 16,500 structures, at least approximately 17,000 structures, at least approximately 17,500 structures, at least approximately 18,000 structures, at least approximately 18,500 structures, at least approximately 19,000 structures, at least approximately 19,500 structures, at least approximately 20,000 structures, at least approximately 21,000 structures, at least approximately 22,000 structures, and any value in between, e.g., from about 16,433 structures to about 21,047 structures). The from first polymers 110, system 100 can decompose the first polymers 110 into fragments and then rebuild the polymer fragments to result in the plurality of second polymers 120. From the reconstructed fragments, second polymers 120 can include at least approximately 100 million polymer structures. The system 100 can be further configured to convert the plurality of second polymers 120 into one or more standardized data strings 130. In some embodiments, the one or more standardized data strings 130 are polymer simplified molecular line entry systems (PSMILES) strings. As will be appreciated, PSMILES strings, such as the one shown in FIG. 1B., may be an example standardized data string format used to represent chemical polymer structures as a common “chemical language”.

Referring back to FIG. 1A, the system 100 can be further configured to separate the standardized data strings 130 into one or more tokens 132. Tokenization of the standardized data strings 130 allows for separation of the standardized data strings 130 into uniquely identifiable symbols while retaining meaningful information. In some embodiments, the system 100 can be configured to tokenize the standardized data strings 130 based at least in part on or more text delimiters. The one or more text delimiters 138, as shown in FIG. 1B, can be one or more characters that separate text strings and can include, without limitation, an asterisk (*), a comma (,), a semicolon (;), quotes (“,′), braces ({ }), pipes (|), slashes (/ \), angle brackets (<, >), and the like. Once the system 100 separates the standardized data strings 130 into one or more tokens 132, the system 100 can be further configured to train a first machine learning algorithm 140 to predict one or more tokens 132 of each of the standardized data strings 130.

As would be appreciated by one of skill in the art, a machine learning algorithm is a subfield within artificial intelligence (AI) that enables computer systems and other related devices to learn how to perform tasks and improve performance of tasks over time. System can incorporate machine learning to perform tasks including, but not limited to, supervised learning algorithms, unsupervised learning algorithms, semi-supervised learning algorithms, reinforcement learning algorithms, and the like. In some embodiments, the first machine learning algorithm 140 can be configured to use natural language processing (NLP) to process PSMILES strings and determine a syntactical relationship between the one or more tokens 132. Resultantly, by determining a syntactical relationship between the one or more tokens 132, the first machine learning algorithm 140 can begin to learn chemical structures of polymers via learning PSMILES strings.

In general, NLP is a machine learning technology that can allow a machine learning algorithm to interpret, manipulate, and comprehend language. With respect to the present technology, the first machine learning algorithm can be configured to use Transformer architecture within NLP technology to predict one or more tokens 132 of a standardized data string 130. As known in the art, Transformer architectures have features such as encoders, decoders, and attention layers which can enable Transformer architectures to surmise understanding of inputs based on position as well as predicting missing parts from inputs, such as in the case of training a machine learning algorithm. Transformer architectures can be employed in several different models such as encoder-only models, decoder-only models, and encoder-decoder models within NLP technology. It should be appreciated that system 100 can incorporate encoder-only, decoder-only models, and encoder-decoder models of the Transformers architecture within the first machine learning algorithm 140. In some embodiments of the present technology, the first machine learning algorithm 140 may be further configured to pass one or more tokens 132 through one or more encoder layers and one or more decoder layers.

As shown in FIGS. 1A and 1C, the first machine learning algorithm 140 may be further configured to create a masked portion 134 of the one or more tokens 132 and an unmasked portion 136 of the one or more tokens for each of the standardized data strings 130. As known in the art, masking can be used during training of a machine learning algorithm to test the ability of the machine learning algorithm to appropriately predict masked elements. In some embodiments, the first machine learning algorithm 140 may embed each token 132 of the unmasked portions 136 with a numerical weight. By giving each token 132 of the unmasked portion 136 a numerical weight, the first machine learning algorithm 140 can then analyze the numerical weights to predict the tokens 132 of the masked portion 134.

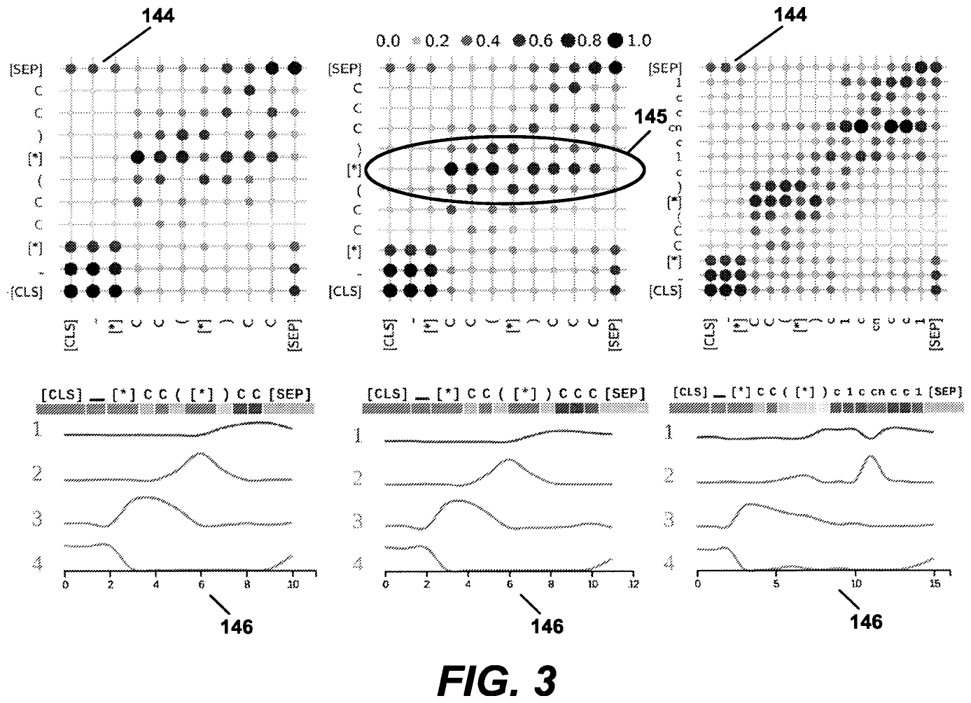

In some embodiments, the first machine learning algorithm 140 may pass each token 132 of the unmasked portion 136 through one or more encoder layers and one or more decoder layers. As each of the tokens pass through the one or more encoder layers and one or more decoder layers, the first machine learning algorithm 140 may update the numerical weight for each token 132 of the unmasked portion 136 to determine a syntactical relationship. Resultantly, the first machine learning algorithm 140 may predict the tokens 132 of the masked portion 134 once a syntactical relationship is determined. In some embodiments, the first machine learning algorithm may create an attention map 144. The attention map 144, an example of which is shown in FIG. 3, can include an attention score 145 for each of the one or more tokens 132. The attention scores 145, which are represented as dots in FIG. 3, may represent the numerical weights that the first machine learning algorithm 140 can give to each of the tokens 132. In some embodiments, the first machine learning algorithm 140 may create one or more neural maps 146, such as those shown in FIG. 3, that may correspond to the attention scores 145 represented in the attention map 144.

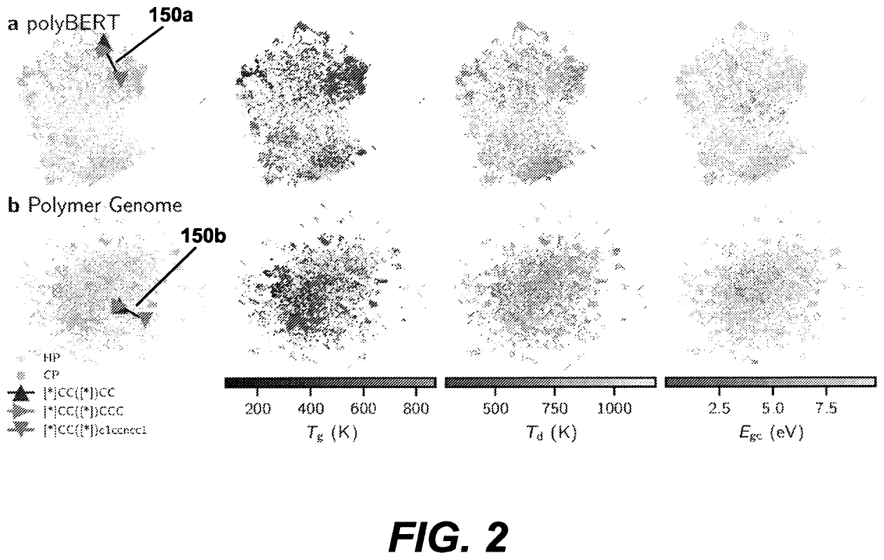

The system 100 can be further configured to compute a unique fingerprint 150 for each of the standardized data strings 130. As shown in FIG. 1C, the unique fingerprint 150 for each of the standardized data strings 130 may be represented as an input vector 152 with 1×N dimensions, wherein N may correspond to the number unique fingerprints 150 which is directly proportional to the number of standardized data strings 130. For comparison, FIG. 2 shows example uniform manifold approximation and projection (UMAP) plots comparing polymer prediction capabilities of handcrafted polymer fingerprints to polymer fingerprints developed by system 100. The unique fingerprint 150a is developed as an output from the first machine learning algorithm 140, as shown in FIGS. 1A and 1C, and demonstrates a chemical pertinence, or relatedness, akin to the unique fingerprint 150b obtained from handcrafted polymer fingerprints, which can be attributed to the training of the first machine learning algorithm. It should be appreciated that the PSMILES string shown in FIG. 2 may be representative of at least one of the plurality of first polymers 110 or the plurality of second polymers 120, in accordance with exemplary embodiments of the present disclosure.

In some embodiments, the system 100 can be further configured to map the unique fingerprints 150 for each standardized data string to a selector vector 162 via a second machine learning algorithm 160. The selector vector 162 can be represented as a binary vector, which can be configured to represent the plurality of polymer properties 170 using a binary number system. It should be appreciated that the selector vector may represent the plurality of polymer properties 170 using other types of number systems including but not limited to hexadecimal, decimal, and the like. It should also be appreciated that the selector vector 162 not having the same dimensions as the input vector 152 does not impact the accuracy of the plurality of polymer properties 170 predicted for each entry of the input vector 152.

In some embodiments, the second machine learning algorithm 160 may be a multitask deep neural network (MTL). As known in the art, MTL is a subfield of machine learning wherein a machine learning algorithm model can be trained to perform multiple tasks at once. MTL can be advantageous when used in conjunction with NLP technologies, such as the Transformers discussed in the present technology, due to the multiple tasks performed being related or having some similarity. With respect to some embodiments of the present disclosure the second machine learning algorithm 160 may use MTL to analyze various features of the unique fingerprints 150 represented as entries in the input vector 152 to predict the plurality of polymer properties 170 for each unique fingerprint 150. The system 100 may also use a single-task or multitask machine algorithms without a neural network as the second machine learning algorithm 160 to analyze various features of the unique fingerprints 150 represented as entries in the input vector 152 to predict the plurality of polymer properties 170 for each unique fingerprint 150. The system 100 may also use a single task machine learning algorithm as the second machine learning algorithm 160 to analyze various features of the unique fingerprints 150 represented as entries in the input vector 152 to predict the plurality of polymer properties 170 for each unique fingerprint 150.

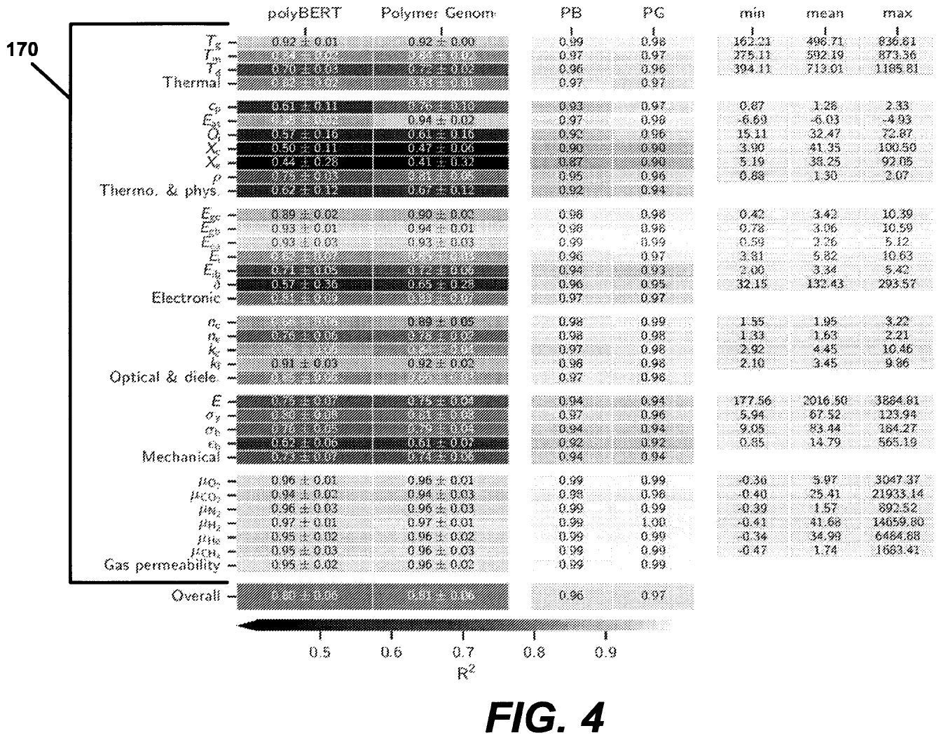

Once the second machine learning algorithm has predicted the plurality of polymer properties 170 for each unique fingerprint 150, the system 100 may be further configured to output the plurality of polymer properties for each unique fingerprint 150. As shown in FIG. 4, the plurality of polymer properties 170 may include but not be limited to the thermal thermodynamic/physical, optical/dielectric, mechanical, or gas permeability characteristics of a given unique polymer fingerprint 150. In some embodiments, the system 100 may be further configured to filter the plurality of polymer properties based at least in part on one or more search parameters. For example, as shown in FIG. 4, the plurality of polymer properties 170 may be represented in a tabular format, wherein parameters can be used to rank each of the unique polymer fingerprints 150 based on their polymer properties 170.

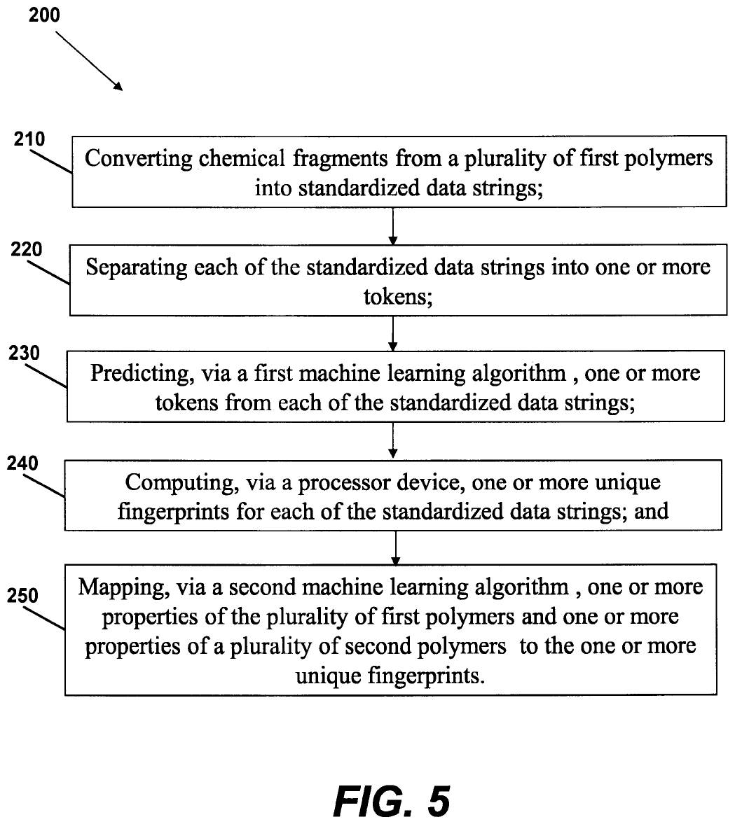

FIG. 5 is a method flow chart, showing a method 200 for predicting polymer properties 170. In some embodiments, the method 200 may include a method step 210 of converting chemical fragments from a plurality of first polymers 110 into standardized data strings 130. The method 200 may further include a method step 220 of separating each of the standardized data strings into one or more tokens 132. The method 200 may further include a method step 230 of predicting, via a first machine learning algorithm 140, one or more tokens 132 from each of the standardized data strings 130. The method 200 may further include a method step 240 of computing, via a processor device, one or more unique fingerprints 150 for each of the standardized data strings 130. The method 200 may further include a method step 250 of mapping, via a second machine learning algorithm 160, one or more properties 170 of the plurality of first polymers 110 and one or more properties 170 of a plurality of second polymers 120 to the one or more unique fingerprints 150.

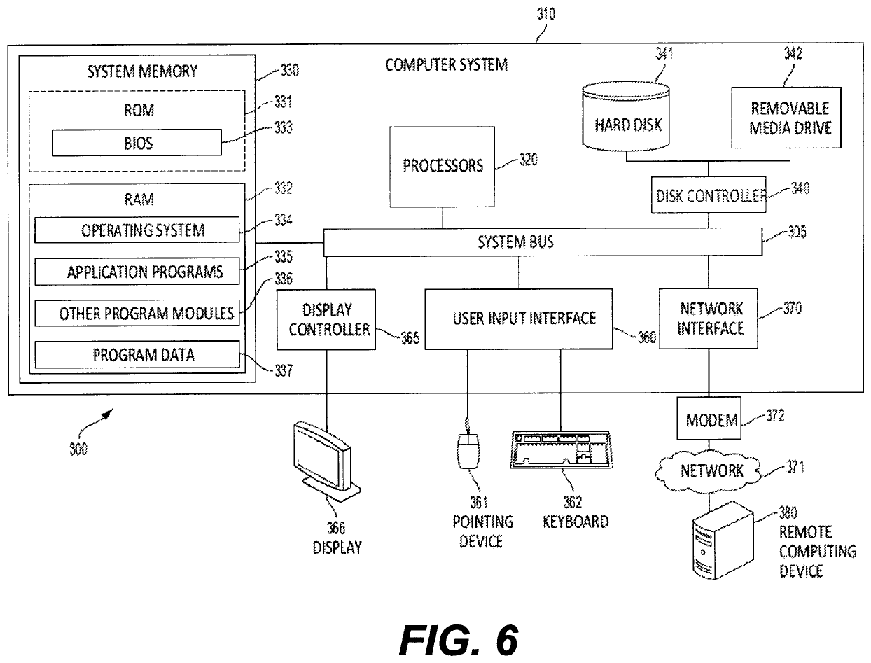

In some embodiments, the system 100 and method 200 can also be implemented in a computing environment, as shown in FIG. 6. FIG. 6 illustrates an exemplary computing environment 300 within which embodiments of the invention may be implemented. For example, this computing environment 300 may be configured to execute a method of placing an item having irregular dimensions. The computing environment 300 may include computer system 310, which is one example of a computing system upon which embodiments of the invention may be implemented. Computers and computing environments, such as computer system 310 and computing environment 300, are known to those of skill in the art and thus are described briefly here.

As shown in FIG. 6, the computer system 310 may include a communication mechanism such as a bus 305 or other communication mechanism for communicating information within the computer system 310. The computer system 310 further includes one or more processors 320 coupled with the bus 305 for processing the information. The processors 320 may include one or more central processing units (CPUs), graphical processing units (GPUs), or any other processor known in the art.

The computer system 310 also includes a system memory 330 coupled to the bus 305 for storing information and instructions to be executed by processors 320. The system memory 330 may include computer readable storage media in the form of volatile and/or nonvolatile memory, such as read only memory (ROM) 331 and/or random access memory (RAM) 332. The system memory RAM 332 may include other dynamic storage device(s) (e.g., dynamic RAM, static RAM, and synchronous DRAM). The system memory ROM 331 may include other static storage device(s) (e.g., programmable ROM, erasable PROM, and electrically erasable PROM). In addition, the system memory 330 may be used for storing temporary variables or other intermediate information during the execution of instructions by the processors 320. A basic input/output system (BIOS) 333 containing the basic routines that help to transfer information between elements within computer system 310, such as during start-up, may be stored in ROM 331. RAM 332 may contain data and/or program modules that are immediately accessible to and/or presently being operated on by the processors 320. System memory 330 may additionally include, for example, operating system 334, application programs 335, other program modules 336 and program data 337.

The computer system 310 also includes a disk controller 340 coupled to the bus 305 to control one or more storage devices for storing information and instructions, such as a hard disk 341 and a removable media drive 342 (e.g., floppy disk drive, compact disc drive, tape drive, and/or solid state drive). The storage devices may be added to the computer system 310 using an appropriate device interface (e.g., a small computer system interface (SCSI), integrated device electronics (IDE), Universal Serial Bus (USB), or Fire Wire).

The computer system 310 may also include a display controller 365 coupled to the bus 305 to control a display 366, such as a cathode ray tube (CRT) or liquid crystal display (LCD), for displaying information to a computer user. The computer system 310 includes an input interface 360 and one or more input devices, such as a keyboard 362 and a pointing device 361, for interacting with a computer user and providing information to the processor 320. The pointing device 361, for example, may be a mouse, a trackball, or a pointing stick for communicating direction information and command selections to the processor 320 and for controlling cursor movement on the display 366. The display 366 may provide a touch screen interface which allows input to supplement or replace the communication of direction information and command selections by the pointing device 361.

The computer system 310 may perform a portion or all of the processing steps of embodiments of the invention in response to the processors 320 executing one or more sequences of one or more instructions contained in a memory, such as the system memory 330. Such instructions may be read into the system memory 330 from another computer readable medium, such as a hard disk 341 or a removable media drive 342. The hard disk 341 may contain one or more datastores and data files used by embodiments of the present invention. Datastore contents and data files may be encrypted to improve security. The processors 320 may also be employed in a multi-processing arrangement to execute the one or more sequences of instructions contained in system memory 330. In alternative embodiments, hard-wired circuitry may be used in place of or in combination with software instructions. Thus, embodiments are not limited to any specific combination of hardware circuitry and software.

As stated above, the computer system 310 may include at least one computer readable medium or memory for holding instructions programmed according to embodiments of the invention and for containing data structures, tables, records, or other data described herein. The term “computer readable medium” as used herein refers to any medium that participates in providing instructions to the processor 320 for execution. A computer readable medium may take many forms including, but not limited to, non-volatile media, volatile media, and transmission media. Non-limiting examples of non-volatile media include optical disks, solid state drives, magnetic disks, and magneto-optical disks, such as hard disk 341 or removable media drive 342. Non-limiting examples of volatile media include dynamic memory, such as system memory 330. Non-limiting examples of transmission media include coaxial cables, copper wire, and fiber optics, including the wires that make up the bus 305. Transmission media may also take the form of acoustic or light waves, such as those generated during radio wave and infrared data communications.

The computing environment 300 may further include the computer system 310 operating in a networked environment using logical connections to one or more remote computers, such as remote computer 380. Remote computer 380 may be a personal computer (laptop or desktop), a mobile device, a server, a router, a network PC, a peer device or other common network node, and typically includes many or all of the elements described above relative to computer system 310. When used in a networking environment, computer system 310 may include modem 372 for establishing communications over a network 371, such as the Internet. Modem 372 may be connected to bus 305 via user network interface 370, or via another appropriate mechanism.

Network 371 may be any network or system generally known in the art, including the Internet, an intranet, a local area network (LAN), a wide area network (WAN), a metropolitan area network (MAN), a direct connection or series of connections, a cellular telephone network, or any other network or medium capable of facilitating communication between computer system 310 and other computers (e.g., remote computer 380). The network 371 may be wired, wireless or a combination thereof. Wired connections may be implemented using Ethernet, Universal Serial Bus (USB), RJ-11 or any other wired connection generally known in the art. Wireless connections may be implemented using Wi-Fi, WiMAX, and Bluetooth, infrared, cellular networks, satellite or any other wireless connection methodology generally known in the art. Additionally, several networks may work alone or in communication with each other to facilitate communication in the network 371.

The embodiments of the present disclosure may be implemented with any combination of hardware and software. In addition, the embodiments of the present disclosure may be included in an article of manufacture (e.g., one or more computer program products) having, for example, computer-readable, non-transitory media. The media has embodied therein, for instance, computer readable program code for providing and facilitating the mechanisms of the embodiments of the present disclosure. The article of manufacture can be included as part of a computer system or sold separately.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

A graphical user interface (GUI), as used herein, comprises one or more display images, generated by a display processor and enabling user interaction with a processor or other device and associated data acquisition and processing functions. The GUI also includes an executable procedure or executable application. The executable procedure or executable application conditions the display processor to generate signals representing the GUI display images. These signals are supplied to a display device which displays the image for viewing by the user. The processor, under control of an executable procedure or executable application, manipulates the GUI display images in response to signals received from the input devices. In this way, the user may interact with the display image using the input devices, enabling user interaction with the processor or other device.

The functions and process steps herein may be performed automatically or wholly or partially in response to user command. An activity (including a step) performed automatically is performed in response to one or more executable instructions or device operation without user direct initiation of the activity.

In any of the embodiments described herein, the system 100 can have data set ranges from at least 35,517 data points for 29 various properties. The properties can represent thermal, thermodynamic & physical, electronic, optical & dielectric, mechanical, and permeability characteristics of chemical polymers, or any other class of measurable or computable properties. The properties can include but not be limited to glass transition temperature (T_{g}), melting temperature (Tₘ), thermal degradation (T_{d}), heat capacity (cₚ), atomization energy (Eₐₜ), limiting oxygen index (Oᵢ), crystallization tendency (Xₑ), crystallization tendency density (ρ), band gap (chain) (E_{gc}), band gap (bulk) (E_{gb}), electron affinity (Eₑₐ), ionization energy (Eᵢ), electronic injection barrier (E_{ib}), cohesive energy density (δ), refractive index (DFT) (n_{c}), refractive index (exp) (nₑ), dielectric constant (DFT) (k_{c}), dielectric constant at frequency “f” (k_{f}), Young's modulus (E), tensile strength at yield (σ_{y}), tensile strength at break (σ_{b}), elongation at break (ϵ_{b}), O₂ gas permeability (ρ_{O2}), N₂ gas permeability (μ_{N2}), CO₂ gas permeability (μ_{CO2}), H₂ gas permeability (μ_{H2}), He gas permeability (μ_{He}), CH₄ gas permeability (μ_{CH4}), and any other property that is measurable or computable. The system 100 can be observed to perform with a high degree of accuracy (R²>80) with respect to predicting properties for polymers, comparatively performing similarly to traditional polymer fingerprinting methods.

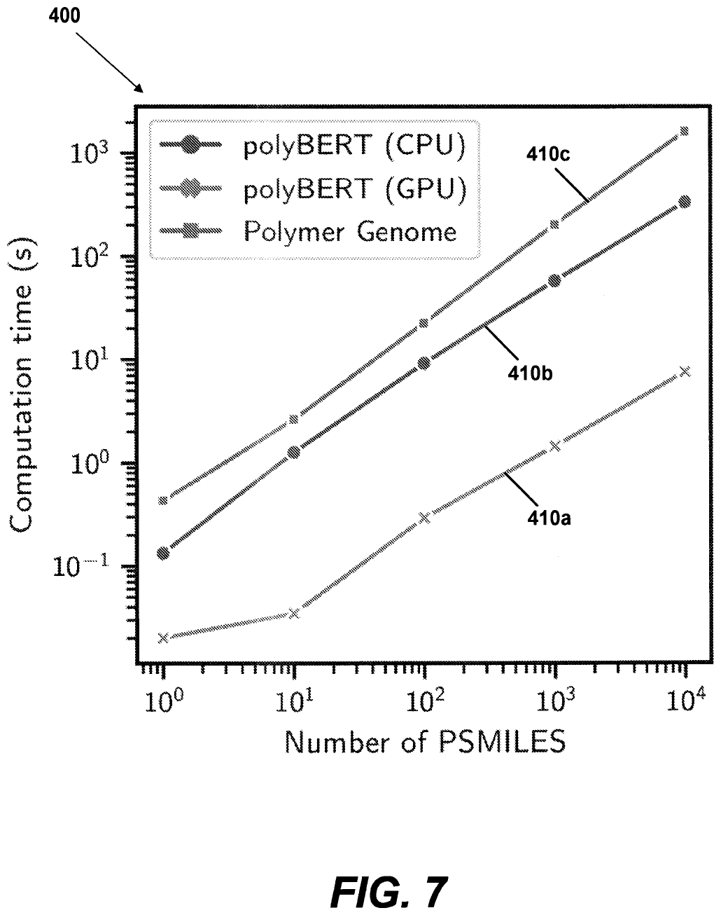

The system 100 can be configured with a speed more than two orders of magnitude (>200 times) faster than any traditional polymer fingerprinting. The system 100 can also be scalable to cloud based computing systems. As shown in FIG. 7, the plot 400 compares computation speeds between the present system 100 against traditional polymer fingerprinting technologies and methods. The GPU plot line 410a and the CPU plot line 410b, as shown in FIG. 7, can be observed to outperform the third plot line 410c, which can be understood as being representative of traditional polymer fingerprinting technologies and methods.

The following examples further illustrate aspects of the present disclosure. However, they are in no way a limitation of the teachings or disclosure of the present disclosure as set forth herein.

### Examples

### Data Sets

FIG. 1A sketches the two-step process for fabricating 100 million hypothetical PSMILES strings. The Breaking Retrosynthetically Interesting Chemical Substructures (BRICS) method (as implemented in RDKit) was used to decompose previously synthesized 13,766 polymers (all monomers of the data set outline in TABLE 1, see below) into 4,424 unique chemical fragments. Random and enumerative compositions of these fragments yield 100 million hypothetical PSMILES strings that were first canonicalized and then use for training polyBERT. The hypothetical PSMILES strings are chemically valid polymers but, mostly, have never been synthesized before.

TABLE 1
Example Training Data Set For Property Predictors

| | | | | Data Points | | |
|---|---|---|---|---|---|---|
| Property (Units) | Symbol | Source^{a} | Data Range | HP | CP | All |

Thermal

| | | | | | | |
|---|---|---|---|---|---|---|
| Glass transition temp. (K) | T_{g} | Exp. | [8e+01, 9e+02] | 5183 | 3312 | 8495 |
| Melting temp. (K) | Tₘ | Exp. | [2e+02, 9e+02] | 2132 | 1523 | 3655 |
| Degradation temp. (K) | T_{d} | Exp. | [3e+02, 1e+03] | 3584 | 1064 | 4648 |

Thermodynamic & Physical

| | | | | | |
|---|---|---|---|---|---|
| Heat capacity (Jg⁻¹K⁻¹) | Cₚ | Exp. | [8e−01, 2e+00] | 79 | 79 |
| Atomization energy (eV atom⁻¹) | Eₐₜ | DFT | [−7e+00, −5e+00] | 390 | 390 |
| Limiting oxygen index (%) | Oᵢ | Exp. | [1e+01, 7e+01] | 101 | 101 |
| Crystallization tendency (DFT) (%) | X_{c} | DFT | [1e−01, le+02] | 432 | 432 |
| Crystallization tendency (exp.) | Xₑ | Exp. | [1e+00, le+02] | 111 | 111 |
| Density (g cm⁻³) | ρ | Exp. | [8e−01, 2e+00] | 910 | 910 |

Electronic

| | | | | | |
|---|---|---|---|---|---|
| Band gap (chain) (eV) | E_{gc} | DFT | [2e−02, 1e+01] | 4224 | 4224 |
| Band gap (bulk) (eV) | E_{gb} | DFT | [4e−01, 1e+01] | 597 | 597 |
| Electron affinity (eV) | Eₑₐ | DFT | [4e−01, 5e+00] | 368 | 368 |
| Ionization energy (eV) | Eᵢ | DFT | [4e+00, 1e+01] | 370 | 370 |
| Electronic injection barrier (eV) | E_{ib} | DFT | [2e+00, 7e+00] | 2610 | 2610 |
| Cohesive energy density | δ | Exp. | [2e+01, 3e+02] | 294 | 294 |

Optical & Dielectric

| | | | | | |
|---|---|---|---|---|---|
| Refractive index (DFT) | n_{c} | DFT | [1e+00, 3e+00] | 382 | 382 |
| Refractive index (exp.) | nₑ | Exp. | [1e+00, 2e+00] | 516 | 516 |
| Dielec. constant (DFT) | k_{c} | DFT | [3e+00, 9e+00] | 382 | 382 |
| Dielec. constant at freq. f^{b} | k_{f} | Exp. | [2e+00, 1e+01] | 1187 | 1187 |

Mechanical

| | | | | | | |
|---|---|---|---|---|---|---|
| Young's modulus (MPa) | E | Exp. | [2e−02, 4e+03] | 592 | 322 | 914 |
| Tensile strength at yield (MPa) | σ_{y} | Exp. | [3e−05, 1e+02] | 216 | 78 | 294 |
| Tensile strength at break (MPa) | σ_{b} | Exp. | [5e−03, 2e+02] | 663 | 318 | 981 |
| Elongation at break (MPa) | ϵ_{b} | Exp. | [3e−01, 1e+03] | 868 | 260 | 1128 |

Permeability

| | | | | | | |
|---|---|---|---|---|---|---|
| O₂ gas permeability (barrer) | μ_{O2} | Exp. | [5e−06, 1e+03] | 390 | 210 | 600 |
| CO₂ gas permeability (barrer) | μ_{CO2} | Exp. | [1e−06, 5e+03] | 286 | 119 | 405 |
| N₂ gas permeability (barrer) | μ_{N2} | Exp. | [3e−05, 5e+02] | 384 | 99 | 483 |
| H₂ gas permeability (barrer) | μ_{H2} | Exp. | [2e−02, 5e+03] | 240 | 46 | 286 |
| He gas permeability (barrer) | μ_{He} | Exp. | [5e−02, 2e+03] | 239 | 58 | 297 |
| CH₄ gas permeability (barrer) | μC_{H4} | Exp. | [4e−04, 2e+03] | 331 | 47 | 378 |
| | | | | 28061 | 7456 | 35517 |

The table above shows an exemplary training data set for the property predictors. The properties are sorted into categories, the category provided at the top of each block. The data set contains 29 properties (dielectric constants k_{f} are available at 9 different frequencies f). HP and CP stand for homopolymer and copolymer, respectively.

Once polyBERT completed its unsupervised learning task using the 100 million hypothetical PSMILES strings, multitask supervised learning maps polyBERT polymer fingerprints to multiple properties to produce property predictors. The property data set in TABLE 1 was used for training the property predictors. The data set contains 28,061 (≈80%) homopolymer and 7,456 (≈20%) copolymer (total of 35,517) data points of 29 experimental and computational polymer properties that pertain to 11,145 different monomers and 1,338 distinct copolymer chemistries, respectively. Each of the 7,456 copolymer data points involved two distinct comonomers at various compositions. The copolymer data points are for random copolymers, which are adequately handled by the adopted fingerprinting strategy (see Methods section). Alternating copolymers are treated as homopolymers with appropriately defined repeat units for fingerprinting purposes. Other flavors of copolymers may also be encoded by adding additional fingerprint components.

polyBERT

polyBERT iteratively ingests 100 million hypothetical PSMILES strings to learn the polymer chemical language, as sketched in FIG. 1B. Using 100 million PSMILES strings is the latest example of training a chemistry-related language model with a large data set and follows the trend of growing data sets in this discipline, with ChemBERTa using 10 million, SMILES-BERT using 18.7 million, and ChemBERTa-2 using 77 million SMILES strings. polyBERT is a DeBERTa model (as implemented in Huggingface's Transformer Python library) with a supplementary three-stage preprocessing unit for PSMILES strings. The DeBERTa model was selected as the foundation of polyBERT because it outperformed other BERT-like models (BERT, ROBERTa, and DistilBERT) in tests and standardized performance tasks. First, polyBERT transforms an input PSMILES string into its canonical form (e.g., [*]CCOCCO[*] to [*]COC[*]) using the canonicalize_psmiles Python package disclosed herein. Second, polyBERT tokenizes canonical PSMILES strings using the SentencePiece tokenizer and a total of 265 tokens. The tokens include common PSMILES characters such as the uppercased and lowercased 118 elements of the periodic table of elements, numbers ranging from 0 to 9, and special characters like [*], (,), =, among others. This ensures that the tokenizer covers the entire PSMILES strings vocabulary. Third, polyBERT masks 15% (default parameter for masked language models) of the tokens to create a self-supervised training task. In this training task, polyBERT is taught to predict the masked tokens using the non-masked surrounding tokens by adjusting the weights of the Transformer encoders (fill-in-the-blanks task). 80 million PSMILES strings were used for training and 20 million PSMILES strings for validation. The validation F1-score was >0.99. This exceptionally good F1-score indicates that polyBERT finds the masked tokens in almost all cases. The total CO₂ emissions for training polyBERT on the hardware are estimated to be 12.6 kgCO₂eq (see CO₂ Emission and Timing section).

The training with 80 million PSMILES strings renders polyBERT an expert polymer chemical linguist who knows grammatical and syntactical rules of the polymer chemical language. polyBERT learns patterns and relations of tokens via the multi-head self-attention mechanism and fully connected feed-forward network of the Transformer encoders. The attention mechanism instructs polyBERT to devote more focus to a small but essential part of a PSMILES string. polyBERT's learned latent spaces after each encoder block are numerical representations of the input PSMILES strings. The polyBERT fingerprint is the average over the token dimension (sentence average) of the last latent space (dotted line in FIG. 1A). The Python package SentenceTransformers was used for extracting and computing polyBERT fingerprints.

### Fingerprints

For acquiring analogies and juxtaposing chemical relevancy, polyBERT fingerprints were compared with the handcrafted Polymer Genome (PG) fingerprints that numerically encode polymers at three different length scales. The PG fingerprint vector for the data set in this work has 945 components and is sparsely populated (93.9% zeros). The reason for this ultra sparsity is that many PG fingerprint components count chemical groups in polymers. A fingerprint component of zero indicates that a chemical group is not present. In contrast, polyBERT fingerprint vectors have 600 components and are fully dense (0% zeros). Fully dense and lower-dimensional fingerprints are often advantageous for ML models whose computation time scales superlinear (O(n^{s}), s>1) with the data set size (n) such as Gaussian process or kernel ridge techniques. Moreover, in the case of neural networks, sparse and high-dimensional input vectors can cause unnecessary high memory load that reduces training and inference speed. The dimensionality of polyBERT fingerprints is a parameter that can be chosen arbitrarily to yield the best training result.

FIG. 2 shows Uniform Manifold Approximation and Projection (UMAP) plots for all homo and copolymer chemistries. The triangles in the first column indicate the coordinates of three selected polymers for polyBERT and PG fingerprints. For both fingerprint types, it was observed that the overlapping triangles are very close, while the non-overlapping triangle is separate. Polymers corresponding to the overlapping triangles, namely poly(but-1-ene) and poly(pent-1-ene), have similar chemistry (different by only one carbon atom), but poly(4-vinylpyridine) represented by a non-overlapping triangle, is different. This chemically intuitive positioning of fingerprints suggests the chemical relevancy of fingerprint distances. The second, third, and fourth columns of FIG. 2 display the same UMAP plots as in the first column. Colored dots indicate the property values of T_{g}, T_{d}, and E_{gc}, while light gray dots show polymer fingerprints with unknown property values. Localized clusters of similar color were observed in each plot pertaining to polymers of similar properties. Although this finding is not surprising for the PG fingerprint because it relies on handcrafted chemical features that purposely position similar polymers next to each other, it is remarkable for polyBERT. With no chemical information and purely based on training on a massive amount of PSMILES strings, polyBERT has learned polymer fingerprints that match chemical intuition. This again shows that polyBERT fingerprints have chemical pertinence and their distances measure polymer similarity (e.g., using the cosine distance metric).

polyBERT learns chemical motifs and relations in the PSMILES strings using the Transformer encoders, each of which includes an attention and feed-forward network layer (see FIG. 1A). FIG. 3 displays the normalized attention maps summed over all 12 attention heads and 12 encoders of polyBERT for the same PSMILES strings as in FIG. 1B. Large dots indicate high attention scores, while small dots show weak attention scores. The attention scores can be interpreted as the importance of knowing the position and type of another token (or chemical motif) and its impact on the current token's latent space. The [CLS] and [September] tokens are auxiliary tokens. The first two tokens indicate the beginning of PSMILES strings and the last token shows the end of PSMILES strings. High attention scores for the [CLS], and first [*] tokens in all panels a to c imply the connection of the auxiliary tokens to the beginning of PSMILES strings. Also, at least intermediate attention scores were observed next and next-to-next neighbors (first and second) off-diagonal elements) for all tokens highlighting the importance of closely bonded neighbors for the polyBERT fingerprint. Another general trend is large attention scores between the second [*] tokens and multiple neighbor tokens across all panels. Moreover, in FIG. 3, large attention scores were found for the en token up to the fourth or fifth neighbor tokens that indicate a strong impact of en to the latent spaces and polyBERT fingerprint, which is expected due to the different nature of the nitrogen atom.

FIG. 3 also shows the non-negative matrix factorizations (four components) of the neuron activations in the feed-forward neural network layers of polyBERT for the same polymers as in panels a to c. The neurons in the feed-forward network layers account for more than 60% of the parameters. Each of the four components represent a set of distinct neurons that are active for specific tokens (x-axes). For example, the fourth set of neurons is active if polyBERT predicts latent spaces for the auxiliary tokens. The third set of neurons fire in the case of the first two C tokens and the first set of neurons are active for side chain c or C atoms, except in the case of the en token, which has its own set of neurons (second set of neurons). In total, the attention layers incorporate positional and relational knowledge and the feed-forward neural network layers disable and enable certain routes through polyBERT. Both factors modulate the polyBERT fingerprints.

Not surprisingly, the computations of polyBERT and PG fingerprints scale nearly linearly with the number of PSMILES strings although their performance (i.e., pre-factor) can be quite different, as shown in the log-log scaled FIG. 7. The computation of polyBERT (GPU) is over two orders of magnitude (215 times) faster than computing PG fingerprints. polyBERT fingerprints may be computed on CPUs and GPUs. Because of the presently large efforts in industry to develop faster and better GPUs, the computation of polyBERT fingerprint is expected to become even faster in the future. Time is very important for high-throughput polymer informatics pipelines that identify polymers from large candidate sets. With an estimate of 0.30 ms/PSMILES for the multitask deep neural networks, the total time using the polyBERT-based pipeline to predict 29 polymer properties sums to 1.06 ms/polymer/GPU.

### Property Prediction

For benchmarking the property prediction accuracy of polyBERT and PG fingerprints, multitask deep neural networks were trained for each property category. Multitask deep neural networks have demonstrated best-in-class results for polymer property predictions while being fast, scalable, and readily amenable if more data points become available. Unlike single-task models, multitask models simultaneously predict numerous properties (tasks) and harness inherent but hidden correlations in data to improve their performance. Such correlation exists, for instance, between T_{g} and Tₘ, but the exact correlation varies across specific polymer chemistries. Multitask models learn and improve from these varying correlations in data. The training protocol of the multitask deep neural networks follows state-of-the-art methods involving five-fold cross-validation and a consolidating meta learner that forecasts the final property values based upon the ensemble of cross-validation predictors.

FIG. 4 shows high R² values for each meta learner (one for each category), suggesting an exceptional prediction performance across all properties. The meta learners were trained on unseen 20% of the data set and validate using 80% of the data set (also used for cross-validation). The reported validation R² values thus only partly measure the generalization performance with respect to the full data set. Meta learners can be conceived as taking decisive roles in selecting the best values from the predictions of the five cross-validation models. The meta learners can be used for all property predictions.

The ultrafast and accurate polyBERT-based polymer informatics pipeline allows the system to predict all 29 properties of the 100 million hypothetical polymers that were originally created to train polyBERT. FIG. 4 shows the minimum, mean, and maximum for each property. Given the vast size of the data set and consequent chemical space of the 100 million hypothetical polymers, the minimum and maximum values can be interpreted as potential boundaries of the total polymer property space. In addition, a data set of this magnitude presents numerous opportunities for obtaining fascinating insights and practical applications. For example, it can be utilized in future studies to establish standardized benchmarks for testing and evaluating ML models in the domain of polymer informatics. The data set may also reveal structure-property information that provides guidance for design rules, helps to identify unexplored areas to search for new polymers, or facilitates direct selection of polymers with specific properties through nearest neighbor searches. A possible future evolution of the data set may also contain subspaces of distinct polymer classes, such as biodegradable or low-carbon polymer classes. However, these aspects are beyond the scope of this study. The data set with 100 million hypothetical polymers including the predictions of 29 properties is available for academic use. The total CO₂ emissions for predicting 29 properties of 100 million hypothetical polymers are estimated to be 5.5 kgCO₂eq.

Other Advantages of polyBERT: Beyond Speed and Accuracy

The feed-forward network as shown in FIG. 1A, which predicts masked tokens during the self-supervised training of polyBERT, enables the mapping of numerical latent spaces (i.e., fingerprints) to PSMILES strings. However, because the system averaged over the token dimension of the last latent space to compute polyBERT fingerprints, it cannot unambiguously map the current fingerprints back to PSMILES strings. A modified future version of polyBERT that provides PSMILES string encoding and fingerprint decoding could involve inserting a dimensionality-reducing layer after the last Transformer encoder. Fingerprint decoders are important elements of design informatics pipelines that invert the prediction pipeline to meet property specifications. The current choice of computing polyBERT fingerprints as pooling averages stems from basic dimensionality reduction considerations require no modification of the DeBERTa architecture.

A second advantage of the polyBERT approach is interpretability. Analyzing the chemical relevancy of polyBERT fingerprints in greater detail can reveal chemical functions and interactions of structural parts of the polymers. As illustrated with the examples of the three polymers in FIG. 3, deciphering and visualizing the attention layers of the Transformer encoders can reveal such information. Saliency methods may also be used to directly explain the relationships between structural parts of the SMILES strings (inputs) and polymer properties (outputs).

FIG. 4 shows the coefficient of determination (R²) averages and standard deviations across the five validation data sets of the cross-validation process for 29 polymer properties. The averages are independent of the data set splits, while the standard deviations show the variance of the prediction performance for the different splits. Smaller standard deviations indicate data sets with homogeneously distributed data points in the learning space. Large standard deviations stem from inhomogeneously distributed data points of usually smaller data sets. Cross-validation is shown to establish an independence of the data set splits for polymer predictions. The prediction accuracy was found to be better for thermal and mechanical properties of copolymers (relative to that for homopolymers) and slightly worse for the gas permeabilities, similar to previous findings. Overall, PG performs best (R²=0.81) but is very closely followed by polyBERT (R²=0.80). This overall performance order of the fingerprint types is persistent with the category averages and properties, except for X_{c}, Xₑ, and ϵ_{b}, where polyBERT slightly outperforms PG fingerprints. polyBERT and PG fingerprints are both practical routes for polymer featurization because their R² values lie close together and are generally high. polyBERT fingerprints have the accuracy of the handcrafted PG fingerprints but are over two orders of magnitude faster.

Yet another advantage of the polyBERT approach is its coverage of the entire chemical space. Molecule SMILES strings are a subset of polymer SMILES strings and differ by only two stars ([*]) symbols that indicate the two endpoints of the polymer repeat unit. polyBERT has no intrinsic limitations or functions that obstruct predicting fingerprints for molecule SMILES strings. The experiments described herein show consistent and well-conditioned fingerprints for molecule SMILES strings using polyBERT that required only minimal changes in the canonicalization routine.

Here, a generalizable, ultrafast, and accurate polymer informatics pipeline is described that is seamlessly scalable on cloud hardware and suitable for high-throughput screening of huge polymer spaces. polyBERT, which is a Transformer-based NLP model modified for the polymer chemical language, is the critical element of the pipeline. After training on 100 million hypothetical polymers, the polyBERT-based informatics pipeline arrives at a representation of polymers and predicts polymer properties over two orders of magnitude faster but at the same accuracy as the best pipeline based on handcrafted PG fingerprints.

The total polymer universe is gigantic, but currently limited by experimentation, manufacturing techniques, resources, and economical aspects. Contemplating different polymer types such as homopolymers, copolymer, and polymer blends, novel undiscovered polymer chemistries, additives, and processing conditions, the number of possible polymers in the polymer universe is truly limitless. Searching this extraordinarily large space enabled by property predictions is limited by the prediction speed. The accurate prediction of 29 properties for 100 million hypothetical polymers in a reasonable time demonstrates that poly-BERT is an enabler to extensive explorations of this gigantic polymer universe at scale. polyBERT paves the pathway for the discovery of novel polymers 100 times faster (and potentially even faster with newer GPU generations) than state-of-the-art informatics approaches—but at the same accuracy as slower handcrafted fingerprinting methods—by leveraging Transformer—based ML models originally developed for NLP. polyBERT finger-prints are dense and chemically pertinent numerical representations of polymers that adequately measure polymer similarity. They can be used for any polymer informatics task that requires numerical representations of polymers such as property predictions (demonstrated here), polymer structure predictions, ML-based synthesis assistants, etc. polyBERT finger-prints have a huge potential to accelerate past polymer informatics pipelines by replacing the handcrafted fingerprints with polyBERT fingerprints. polyBERT may also be used to directly design polymers based on fingerprints (that can be related to properties) using polyBERT's decoder that has been trained during the self-supervised learning. This, however, requires retraining and structural updates to polyBERT and is thus part of a future work.

### Example Methods

### PSMILES Canonicalization

The string representations of homopolymer repeat units in this work are PSMILES strings. PSMILES strings follow the SMILES syntax definition but use two stars to indicate the two endpoints of the polymer repeat unit (e.g., [*]CC[*] for polyethylene). The raw PSMILES syntax is non-unique; i.e., the same polymer may be represented using many PSMILES strings; canonicalization is a scheme to reduce the different PSMILES strings of the same polymer to a single unique canonicalized PSMILES string. polyBERT requires canonicalized PSMILES strings because polyBERT fingerprints change with different writings of PSMILES strings. In contrast, PG fingerprints are invariant to the way of writing PSMILES strings and, thus, do not require canonicalization. FIG. 6 shows three variances of PSMILES strings that leave the polymer unchanged. The translational variance of PSMILES strings allows to move the repeat unit window of polymers (cf., white and red box). The multiplicative variance permits to write polymers as multiples of the repeat unit (e.g., two-fold repeat unit of Nylon 6), while the permutational variance stems from the SMILES syntax definition and allows syntactical permutations of PSMILES strings that leave the polymer unchanged.

As described herein, the canonicalize PSMILES Python package was developed to find the canonical form of PSMILES strings in four steps; (i) it finds the shortest PSMILES string by searching and removing repetition patterns, (ii) it connects the polymer endpoints to create a periodic PSMILES string, (iii) it canonicalizes the periodic PSMILES string using RDKit's canonicalization routines, (iv) it breaks the periodic PSMILES string to create the canonical PSMILES string.

### Polymer Fingerprinting

Fingerprinting converts geometric and chemical information of polymers (based upon the PSMILES string) to machine-readable numerical representations in the form of vectors. These vectors are the polymer fingerprints and can be used for property predictions, similarity searches, or other tasks that require numerical representations of polymers.

The polyBERT fingerprints were compared with the handcrafted Polymer Genome (PG) polymer fingerprints. PG fingerprints capture key features of polymers at three hierarchical length scales. At the atomic scale (1st level), PG fingerprints track the occurrence of a fixed set of atomic fragments (or motifs). The block scale (2nd level) uses the Quantitative Structure-Property Relationship (QSPR) fingerprints for capturing features on larger length-scales as implemented in the cheminformatics toolkit RDKit. The chain scale (3rd level) fingerprint components deal with “morphological descriptors” such as the ring distance or length of the largest side-chain.

As discussed the composition-weighted polymer fingerprints were summed to compute copolymer fingerprints F=P_{N} F_{c}, where N is the number of comonomers in the copolymer, F is the comonomer fingerprint vector, and c is the fraction of the comonomer. This approach renders copolymer fingerprints invariant to the order in which one may sort the comonomers and satisfies the two main demands of uniqueness and invariance to different (but equivalent) periodic unit specifications. While the current fingerprinting scheme is most appropriate for random copolymers, other copolymer flavors may be encoded by adding additional fingerprint components. Contrary to homopolymer fingerprints, copolymer fingerprints may not be interpretable (e.g., the composition-weighted sum of the fingerprint component “length of largest side-chain” of two homopolymers has no physical meaning).

### Multitask Neural Networks

Multitask deep neural networks simultaneously learn multiple polymer properties to utilize inherent correlations of properties in data sets. The training protocol of the concatenation-conditioned multitask predictors follows state-of-the-art techniques involving five-fold cross-validation and a meta learner that forecasts the final property values based upon the ensemble of cross-validation predictors. After shuffling, the data set was split into two parts and use 80% for the five cross-validation models and for validating the meta learners 20% of the data set is used for training the meta learners. The Hyperband method of the Python package KerasTuner was used to fully optimize all hyperparameters of the neural networks, including the number of layers, number of nodes, dropout rates, and activation functions. The Hyperband method finds the best set of hyperparameters by minimizing the Mean Squared Error (MSE) loss function. Data set stratification of all splits was performed based on the polymer properties. The multitask deep neural networks are implemented using the Python API of TensorFlow.

### CO₂ Emission and Timing

Experiments were conducted using a private infrastructure, which has an estimated carbon efficiency of 0.432 kgCO₂eqkWh⁻¹. A total of 31 hours of computations were performed on four Quadro-GP100-16 GB (thermal design power of 235 W) for training polyBERT. Total emissions are estimated to be 12.6 kgCO₂eq. About 8 hours of computations on four GPUs were necessary for training the cross-validation and meta learner models with an estimated emission of 3.3 kgCO₂eq for polyBERT and Polymer Genome fingerprints, respectively. The total emissions for predicting 29 properties for 100 million hypothetical polymers are estimated to be 5.5 kgCO₂eq, taking a total of 13.5 hours. Estimations were conducted using a Machine Learning Impact calculator.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A method comprising:
preprocessing polymer simplified molecular input line end system (“PSMILES”) strings;
feeding the preprocessed PSMILES strings to an encoder-only transformer chemical language model;
fingerprinting at least a portion of an output of the encoder-only transformer chemical language model; and
predicting polymer properties from the fingerprinted output.

2. The method of claim 1, wherein the method is an integrated end-to-end completely machine-driven polymer informatics pipeline.

3. The method of claim 1, wherein the preprocessing comprises:
canonicalizing the PSMILES strings into standardized data strings;
tokenizing at least a portion of the standardized data strings; and
masking at least a portion of the tokenized standardized data strings.

4. The method of claim 1 further comprising:
creating a training set of polymers;
training the encoder-only transformer chemical language model with the training set; and
yielding a machine-driven polymer fingerprinting model for the fingerprinting.

5. The method of claim 2, wherein:
the preprocessing comprises:
converting chemical fragments from a set of first polymers into standardized data strings by representing set of first polymers into the PSMILES strings;
separating the PSMILES strings into one or more tokens; and
predicting, via a first machine learning algorithm, one or more tokens from the PSMILES strings;
the fingerprinting comprises:
computing, via a processor device, one or more unique fingerprints for the PSMILES strings; and
mapping, via the encoder-only transformer chemical language model, one or more properties of the set of first polymers and one or more properties of set of second polymers to the one or more unique fingerprints; and
the predicting comprises predicting, via encoder-only transformer chemical language model, one or more properties for a new polymer based at least in part on one or more of the properties of the set of second polymers and one or more of the properties of set of second polymers.

6. The method of claim 5, wherein:
the representing the set of first polymers into the PSMILES strings comprises canonicalizing the PSMILES string to create the PSMILES strings for the set of first polymers;
the separating the PSMILES strings into one or more tokens comprises parsing through the PSMILES strings using one or more text delimiters comprises tokenizing the PSMILES strings based at least in part on the one or more text delimiters; and
the predicting, via the first machine learning algorithm, the one or more tokens from the PSMILES strings comprises creating a masked portion of one or more of the tokens and an unmasked portion of one or more of the tokens for the PSMILES strings.

7. The method of claim 6, wherein creating the masked portion and the unmasked portion within the PSMILES strings comprises:
embedding one or more of the tokens of the unmasked portion with a numerical weight; and
predicting the masked portion based on the numerical weight for one or more of the tokens of the unmasked portion.

8. The method of claim 7, embedding one or more of the tokens of the unmasked portion with a numerical weight comprises:
passing one or more of the tokens of the unmasked portion through one or more neural encoder layers and one or more neural decoder layers; and
updating the numerical weight through one or more of the neural decoder layers and one or more of the neural decoder layers for one or more of the tokens of the unmasked portion.

9. The method of claim 8, updating the numerical weight through one or more of the neural decoder layers and one or more of the neural decoder layers for one or more of the tokens of the unmasked portion comprises:
determining a syntactical relationship between one of more of the tokens within the PSMILES strings by creating an attention map for one of more of the tokens;
wherein the attention map is configured to plot an attention score for one of more of the tokens.

10. The method of claim 5, wherein the mapping, via encoder-only transformer chemical language model, the one or more properties of the set of first polymers and the one or more properties of set of second polymers to the one or more unique fingerprints comprises:
receiving an input vector of one of more of the unique fingerprints; and
mapping the input vector with the polymer properties via a selector vector;
wherein the selector vector is a binary vector configured to represent the polymer properties using a binary number format.

11. The method of claim 5 further comprising:
mapping the polymer properties based at least in part on one of more of the unique fingerprints; and
outputting one of more of the polymer properties for one of more of the unique fingerprints by filtering the output of one or more polymer properties based at least in part on one or more search parameters.

12. A system for predicting polymer properties using the method of claim 1 comprising:
a processor device configured to:
convert chemical fragments from a set of first polymers into a set of second polymers different than the first polymers;
convert the set of second polymers into the PSMILES strings;
separate the PSMILES strings into one or more tokens; and
compute one or more unique fingerprints for the PSMILES strings with the encoder-only transformer chemical language model.

13. The system of claim 12, wherein the processor device is further configured to:
parse through the PSMILES strings using one or more text delimiters; and
tokenize the PSMILES strings based at least in part on one of more of the text delimiters.

14. The system of claim 13, wherein the processor device is further configured to:
train a machine learning algorithm configured to predict one or more tokens of the PSMILES strings.

15. The system of claim 14, wherein the machine learning algorithm is further configured to:
use natural language processing (NLP) on one of more of the tokens of each of the PSMILES strings; and
create a masked portion of one of more of the tokens and an unmasked portion of one of more of the tokens for the PSMILES strings.

16. The system of claim 15, wherein the machine learning algorithm is further configured to:
embed one of more of the tokens of the unmasked portion with a numerical weight;
analyze the numerical weight for one of more of the tokens of the unmasked portion to predict the masked portion.

17. The system of claim 16, wherein the machine learning algorithm is further configured to:
pass one of more of the tokens of the unmasked portion through one or more neural encoder layers and one or more neural decoder layers;
update the numerical weight through one of more of the neural encoder layers and one or more of the neural decoder layers for one of more of the tokens of the unmasked portion;
determine a syntactical relationship between one of more of the tokens within the PSMILES strings; and
create an attention map for one of more of the tokens;
wherein the attention map is a plot of an attention score for one of more of the tokens.

18. A system for predicting polymer properties comprising:
a processor device configured to:
receive an input vector;
map via a machine learning algorithm each entry of the input vector with a selector vector indicative of polymer properties; and
output polymer properties for one or more of the entries of the input vector;
wherein one or more of the entries of the input vector is indicative of a unique fingerprint for one or more polymers.

19. The system of claim 18, wherein the machine learning algorithm is a multitask deep neural network.

20. The system of claim 19, wherein the processor device is further configured to filter the output of one or more of the polymer properties based at least in part on one or more search parameters; and
wherein the selector vector is a binary vector configured to represent the polymer properties using a binary number format.
